(19) United States
(12) Patent Application Publication
(10) Pub. No.: US 2016/0074444 A1
(43) Pub. Date: Mar. 17, 2016

(54) LACTOBACILLUS RHAMNOSUS STRAIN FOR REGULATING LIPID METABOLISM

(71) Applicants: COMPAGNIE GERVAIS DANONE, Paris (FR); INSTITUT NATIONAL DE LA RECHERCHE AGRONOMIQUE, Paris (FR)

(72) Inventors: Elsa Jacouton, Meudon (FR); Hervé Blottiére, Magny Les Hameaux (FR); Tamara Smokvina, Orsay (FR); Joel Doré, Vitry-Sur-seine (FR); Johan Van Hylckama Vlieg, Marly Le Roi (FR)

(73) Assignees: INSTITUT NATIONAL DE LA RECHERCHE AGRONOMIQUE, Paris (FR); COMPAGNIE GERVAIS DANONE, Paris (FR)

(21) Appl. No.: 14/784,760

(22) PCT Filed: Apr. 18, 2014
(86) PCT No.: PCT/IB2014/060841

§ 371 Date: Oct. 15, 2015

(30) Foreign Application Priority Data
Apr. 19, 2013 (FR) 13 53600

(51) Int. Cl. A61K 35/747; A23L 1/30; A23C 9/123; A61K 9/00

(52) U.S. Cl. CPC A61K 35/747; A61K 9/0053; A23L 1/3014; A23C 9/1234; A61K 2035/115

(57) ABSTRACT

The invention relates to the use of a strain of Lactobacillus rhamnosus for preventing or treating pathologies associated with an excessive accumulation of body fat. This strain is preferably administered orally, in particular in the form of a food or a food supplement.

The invention relates to the use of probiotics for regulating lipid metabolism.

Lipid metabolism plays an important role in energy homeostasis. The energy from food is stored in adipocytes in the form of lipid stores which are used where necessary to meet the energy needs of the body. A calorie intake which exceeds the calories expended causes the accumulation of body fat, resulting in excess weight and, in the long term, in obesity. Excess weight is defined by a body mass index (BMI) greater than or equal to 25, and obesity by a BMI greater than or equal to 30.

Obesity constitutes an important factor in the occurrence of diseases such as hypertension, type II diabetes, cardiovascular diseases, liver diseases and certain cancers, and is rapidly becoming a major public health problem. The number of obese individuals throughout the world has more than doubled since 1980. In 2008, more than 1.4 billion adults were overweight, among them, more than 200 million men and almost 300 million women were obese.

It is generally acknowledged that, among the main causes of the current frequency of obesity and of metabolic disorders which are associated therewith, are a reduced physical activity and a diet rich in fats and sugar.

However, the inter-individual differences observed in terms of propensity to accumulate fat and gain weight also correlate with other factors, such as genetic background, health conditions, medical treatments, age or lack of sleep.

Among the numerous genes involved in obesity is the gene encoding the ANGPTL4 (angiopoietin like 4) protein, also called FIAF (fasting induced adipose factor), PGAR (PPAR-γ induced angiopoietin-related protein) or HFARP (hepatic fibrinogen/angiopoietin related protein). This protein was initially discovered in 2000 (YOON et al., Mol Cell Biol, 20, 5343-9, 2000; KERSTEN et al., J Biol Chem, 275, 28488-93, 2000; KIM et al., Biochem J, 346 Pt 3, 603-10, 2000). It is well conserved in mammals, with 77% sequence homology between humans (hANGPTL4) and mice (mANGPTL4) (KIM et al., Biochem J, 346 Pt 3, 603-10, 2000). It is predominantly expressed in the liver, the adipose tissue (AT) and the intestine, and its expression is increased by fasting and physical exercise. ANGPTL4 is a target gene of nuclear PPARs (peroxisome proliferator-activated receptors), in particular PPAR-γ in the AT and the colon, and PPAR-α in the liver and the small intestine.

Among the physiological effects attributed to ANGPTL4 is in particular its role in lipid and glucose metabolism. In particular, ANGPTL4 inhibits the activity of lipoprotein lipase (LPL) by promoting the dissociation of the active dimers of LPL into inactive monomers (SUKONINA et al., Proc Natl Acad Sci USA, 103, 17450-5, 2006), thereby resulting in the mobilization of triglycerides as an energy source in favor of the peripheral organs, and the limitation of excessive fat storage in organs such as the adipose tissue, the heart and the macrophages.

It has also been observed that ANGPTL4 improves glucose tolerance in obese mice with type II diabetes (XU et al., Proc Natl Acad Sci USA, 102, 6086-91, 2005), but induces, moreover, short-term hyperlipidemia in this animal model. In humans, an inverse correlation between plasma ANGPTL4 levels, adiposity or increased blood glucose has been reported (ROBCIUC et al., J Lipid Res, 51, 824-31, 2010; ROBCIUC et al., J Lipid Res, 52, 1575-82, 2011).

Other effects have been attributed to the ANGTPL4 protein, such as an anorexigenic effect (KIM et al., Diabetes, 59, 2772-80, 2010) or a decrease in the inflammation caused by saturated food lipids (LICHTENSTEIN et al., Cell Metab, 12, 580-92, 2010).

It has been observed that there is a strong inter-individual variation in the plasma ANGPTL4 level (ROBCIUC et al., J Lipid Res, 51, 824-31, 2010), but that the genetic background plays only a minor role in this variability (ROBCIUC et al., J Lipid Res, 52, 1575-82, 2011).

Moreover, it has been shown that the intestinal microbiota plays an important role in controlling ANGPTL4 expression in the small intestine. The level of ANGPTL4 expression in the small intestine is higher in axenic mice than in conventional mice, and the colonization of axenic mice reduces its level of expression (BACKHED et al., Proc Natl Acad Sci USA, 101, 15718-23, 2004). These modulations of ANGPTL4 expression correlate with the levels of activity of lipoprotein lipase (LPL) in the adipose tissue and the heart, and consequently with fat storage in the adipose tissue. More recently, it has been observed that commensal or probiotic bacteria can modulate ANGPTL4 expression. Thus, Lactobacillus paracasei F19 induces the intestinal expression of ANGPTL4 (PPAR-dependent) resulting, in vivo, in a decrease in fat mass (ARONSSON et al., PLoS One, 5, 2010). In the same way, a commensal bacterium, Clostridium tyrobutyricum, induces a strong expression of ANGPTL4 during the colonization of axenic mice (KORECKA et al., Am J. Physiology Gastrointest Liver Physiol, in press, 2013). The mechanisms involved in this bacteria regulation are at the current time poorly elucidated: in certain cases, they may be direct; in other cases, they may be mediated by secreted factors or metabolites such as short-chain fatty acids (SCFAs), or hydrogen peroxide (H₂O₂) (KORECKA et al., Am J. Physiology Gastro, in press, 2013; GROOTAERT et al., Environ Microbiol, 13, 1778-89, 2011).

More generally, the role of the intestinal microbiota in obesity is the subject of increasing interest, and it has been suggested that the manipulation thereof using prebiotics, probiotics or symbiotics might help to reduce obesity and associated metabolic disorders (MALLAPPA et al., Indian J Endocrinol Metab, 16, 20-7, 2012; DELZENNE et al., Nat Rev Endocrinol, 7, 639-46, 2011).

It has been reported that certain probiotic strains make it possible to reduce fat accumulation and/or metabolic disorders associated with obesity. Effects of these various probiotics appear, however, to be specific for the strain concerned, and mediated by mechanisms which differ from one strain to the other.

In this context, the inventors have tested various lactobacillus strains in order to determine their effects on expression of the ANGPTL4 protein, and have identified, among them, a Lactobacillus rhamnosus strain capable of significantly increasing this expression. It is the Lactobacillus rhamnosus strain deposited according to the Treaty of Budapest on May 19, 2010, under number I-4317, with the CNCM (Collection Nationale de Cultures de Microorganismes [French National Collection of Microorganism Cultures], 25 Rue du Docteur Roux, Paris). This strain is also described in PCT application WO 2011/148219.

Consequently, a subject of the present invention is the use of the Lactobacillus rhamnosus strain CNCM I-4317 or of a composition containing said strain, for preventing or reducing excessive fat accumulation in the adipocytes and the metabolic and/or inflammatory consequences ensuing therefrom, such as insulin resistance in a subject or chronic inflammation. Preferably, said subject is a mammal, which may be an animal or a human.

In particular, the present invention encompasses the Lactobacillus rhamnosus strain CNCM I-4317 or a composition containing said strain, for use as a medicament in the treatment, prevention or attenuation of a pathological condition due to an excessive accumulation of body fat and/or to insulin resistance.

Examples of pathological conditions resulting from excessive accumulation of body fat are excess weight, obesity and the disorders associated therewith, such as metabolic syndrome, type II diabetes, nonalcoholic hepatic steatosis, hypertension, chronic inflammation, etc.

The present invention also encompasses the non-therapeutic use of the CNCM I-4317 strain for preventing or reducing excessive accumulation of body fat in a subject for esthetic purposes.

For the implementation of the present invention, the Lactobacillus rhamnosus strain CNCM I-4317 can be used in the form of whole bacteria which may be living or non-living, for example heat-inactivated. Alternatively, it can be used in the form of a bacterial lysate, or in the form of bacterial fractions; the bacterial fractions suitable for this use are in particular culture supernatents of the CNCM I-4317 strain.

The compositions used in the present invention may be in any form suitable for administration, in particular oral administration. This includes, for example, semi-solid, liquid and powder formulations.

When the bacteria are in the form of living bacteria, the composition can typically comprise from 10⁵ to 10¹³ colony-forming units (cfu), in particular at least 10⁶ cfu, preferably at least 10⁷ cfu, advantageously at least 10⁸ cfu, and entirely preferably at least 10⁹ cfu per gram of dry weight of the composition. In the case of a liquid composition, this generally corresponds to from 10⁴ to 10¹² cfu, preferably at least 10⁵ cfu, more preferably at least 10⁶ cfu, advantageously at least 10⁷ cfu, and entirely preferably at least 10⁹ cfu/ml.

The compositions which are preferred for use in the present invention are nutritional compositions, including food products and in particular milk products. These nutritional compositions also comprise food supplements and functional foods. A “food supplement” denotes a product produced from compounds normally used in food products, but which is in the form of tablets, powder, capsules, a potion or any other form not normally associated with foods, and which has beneficial effects on the health. A “functional food” is a food which, in addition to its nutritional properties, also has beneficial effects on the health. In particular, the food supplements and the functional foods can have a protective or curative physiological effect against a disease, for example against a chronic disease.

Other examples of compositions suitable for use in the present invention are pharmaceutical or cosmetic compositions.

The compositions of the invention can also comprise, in addition to the CNCM I-4317 strain, one or more other strain(s) of lactic acid bacteria, which may or may not be probiotic, for example one or more bacterial strain(s) chosen from the genera Lactobacillus, Lactococcus, Streptococcus, and bifidobacteria. In particular this (these) other strain(s) can include one or more Streptococcus thermophilus strain(s) and/or one or more Lactobacillus bulgaricus strain(s).

The present invention will be understood more clearly by means of the further description which follows, which refers to examples illustrating the effect of the CNCM I-4317 strain on the expression of the ANGPTL4 protein.

### EXAMPLE 1

### Induction of ANGPTL4 Gene Expression by Lactic Acid Bacteria in Intestinal Epithelial Cells

The effect of several bacterial strains belonging to the L. paracasei and L. rhamnosus species on ANGPTL4 gene expression was measured in HT29 colon cancer cells, and compared with that of rosiglitazone (PPARγ receptor agonist, known to very strongly increase ANGPTL4 expression).

### Cell Cultures

The HT29 cells were cultured in DMEM medium (Lonza®) supplemented with 20% of fetal calf serum (FCS) (Lonza®), 2 mM of glutamine (Sigma®), 1× of nonessential amino acids (NEAAs) (Invitrogen®) and 50 U/ml of a penicillin/streptomycin mixture (Lonza®) in an environment containing 10% CO₂ at 37° C.

The cells were detached with trypsin-versene EDTA (Lonza®) and deposited in a 6-well plate. 625 000 cells were deposited per well and cultured for 48 h in antibiotic-free DMEM medium.

The medium was changed before bringing the cells into contact, for 6 h, with the bacterial suspensions, DMEM medium (negative control) or rosiglitazone (positive control). The cell cultures were then rinsed with PBS (Lonza®) before extracting the RNA therefrom.

The rosiglitazone (Cayman chemical®) was prepared in DMSO and diluted in the cell culture medium. This preparation was brought into contact with the cells at a final concentration of 10 μM.

### Bacterial Cultures

A preculture of the Lactobacillus paracasei and rhamnosus strains was carried out in MRS (Man, Rogosa and Sharpe medium—Oxoid® CM0359) overnight. The bacteria were then inoculated at 0.2% of the final volume and cultured for 6 h.

1 ml of each bacterium was centrifuged at 6000 g for 10 minutes and washed with PBS. The bacterial pellets were taken up in the antibiotic-free DMEM cell medium at a final OD₆₀₀ (optical density) of 0.1, which corresponds to approximately 2 to 7×10⁷ CFU/ml.

The bacterial suspensions were then added to the cells at 20% of the final volume.

### RNA Extraction and cDNA Synthesis

The cellular RNA extraction was carried out with the RNeasy mini kit (Qiagen®) according to the supplier's instructions. The RNA was assayed using nanodrop and an agarose gel was run to determine the quality of the RNAs.

The cDNA synthesis was carried out using 1 μg final concentration of RNA by means of the High capacity cDNA reverse transcription kit (Applied Biosystems®) and the T100 Thermal Cycler machine (Biorad®) according to the supplier's instructions.

The cDNAs obtained were assayed using nanodrop and preparations at 100 ng/μl were produced in nuclease-free water (Ambion®) for the RTqPCR.

### RT-qPCR

The RTqPCRs were carried out using the Taqman® gene expression master reagents (Applied Biosystems®) with the ABI Prism 7000 machine (Applied Biosystems®) according to the supplier's instructions.

The statistical analyses were carried out with the Graph Pad Prism 5 software (version 8.0).

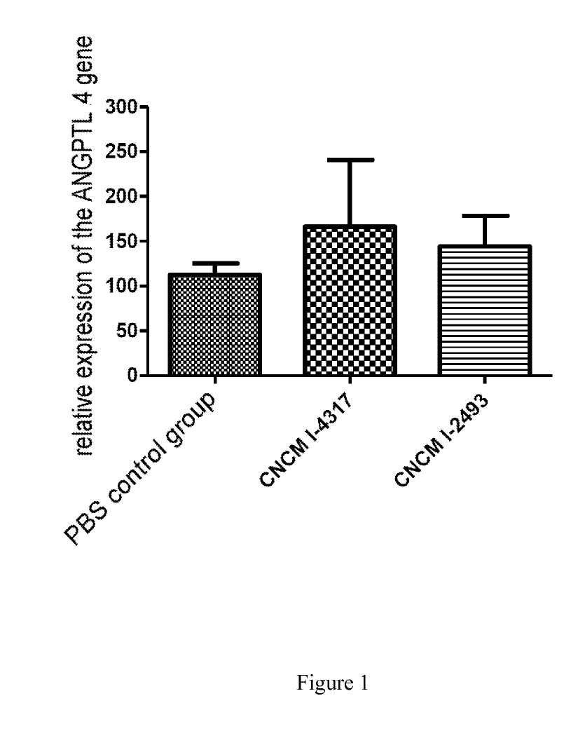

The results are given in FIG. 1.

Legend of FIG. 1: along the x-axis, strains tested; along the y-axis, ANGPTL4 expression, standardized relative to the β-actin control gene. p<0.001 indicates a significant difference (ANOVA test) compared with the DMEM negative control.

These results show that the CNCM I-4317 strain is capable of inducing a significant increase, of approximately 8 times, in the expression of the ANGPTL4 gene.

### EXAMPLE 2

### Study of ANGPTL4 Gene Expression in the Distal Small Intestine of Axenic Mice having Ingested the CNCM I-4317 Bacterium

### Materials and Methods

Germ-free (GF) C57BL/6 mice, 7 to 11 weeks old, were kept on a standard diet (R 03-40, SAFE). The mice were colonized by gavage with 10⁹ CFU/ml of bacteria (fresh cultures suspended in PBS). They were isolated in 3 separate incubators according to the gavage received (N=7 mice given Lactobacillus rhamnosus CNCM I-4317 by gavage, N=6 mice given CNCM I-2493 by gavage and N=5 mice given PBS by gavage). After 2 weeks of stabilization, the mice were sacrificed by cervical dislocation. The colonization was confirmed by counting in the feces API 50CH strip (Biomérieux®). The tissues (small intestine, colon) were removed and stored in RNAlater (stabilization reagent) at −80° C. The RNA from the tissues was extracted using the RNeasy mini kit (Qiagen) and the cDNA was synthesized from 1 μg of RNA using the High Capacity cDNA Reverse Transcription kit (Applied Biosystems). The cDNAs were diluted to 20 ng/μl and the RTqPCRs were carried out with a final volume of 25 μl using Taqman probes (Life Technologies) and the ABI Prism 7700 thermocycler machine (Applied Biosystem) according to the supplier's recommendations. The results were standardized using 2 control genes (GAPDH and β-actin).

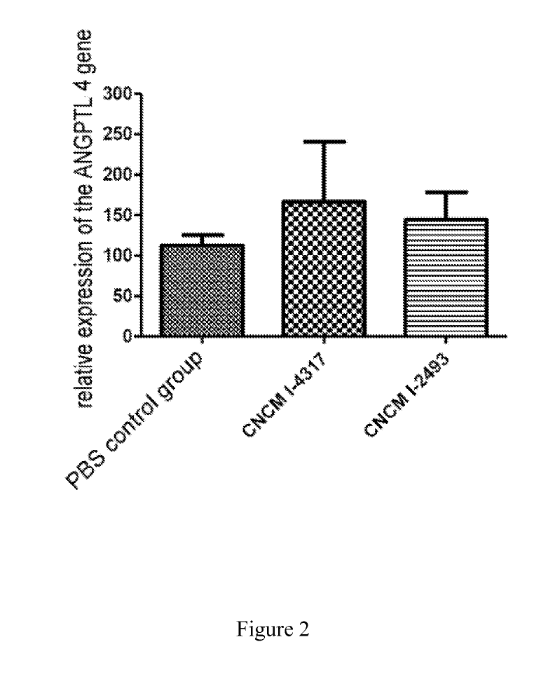

The results are given in FIG. 2.

Legend of FIG. 2: along the x-axis, strains tested; along the y-axis, ANGPTL4 expression, standardized relative to the β-actin and GAPDH control genes.

The analysis of ANGPTL4 gene expression in a murine model demonstrated a tendency of the CNCM I-4317 bacterium to induce this expression in the distal small intestine.

1. A method of preventing or treating a pathological condition resulting from an excessive accumulation of body fat and/or from insulin resistance in a subject, comprising: administering a medicament comprising an effective amount of a Lactobacillus rhamnosus strain deposited at the CNCM (Collection Nationale De Cultures De Microorganismes) under Accession number I-4317 to the subject.

2. The method of claim 1, wherein said pathological condition is selected from the group consisting of excess weight, obesity, and diseases resulting therefrom.

3. The method of claim 1, wherein said medicament is formulated for oral administration.

4. The method of claim 1, wherein said strain is contained in an orally administrable composition.

5. The method of claim 4, wherein said composition is a food or a food supplement.

6. The method of claim 4, wherein said composition is a milk product.
